(19) United States
(12) Patent Application Publication
(10) Pub. No.: US 2009/0297399 A1
(43) Pub. Date: Dec. 3, 2009

(54) Photocatalytic Fog Disseminating System for Purifying Air and Surfaces

(75) Inventors: Robert E. RYAN, Diamondhead, MS (US); Bruce Davis, Long Beach, MS (US); George A. May, Slidell, LA (US); Lauren W. Underwood, Diamondhead, MS (US)

Correspondence Address: CROWELL & MORING LLP;INTELLECTUAL PROPERTY GROUP, P.O. BOX 14300, WASHINGTON, DC, 20044-4300, US

(73) Assignee: Institute of Technology Development, Stennis Space Center, MS (US)

(21) Appl. No.: 12/130,494

(22) Filed: May 30, 2008

(51) Int. Cl. A61L 9/14; A61L 2/23; G05D 99/00

(52) U.S. Cl. 422/4; 422/186; 422/105; 422/122; 422/29

(57) ABSTRACT

A photocatalytic fog disseminating system is used to disperse and activate photocatalytic particles. The photocatalytic particles preferably are nanoscale particles of titanium oxide. An aqueous suspension of the photocatalytic particles is dispersed by the system as an aerosol, and a photon source is used to excite the dispersed particles in order to initiate photocatalytic redox reactions that degrade airborne or sedentary organic impurities that come into contact with the aerosol.

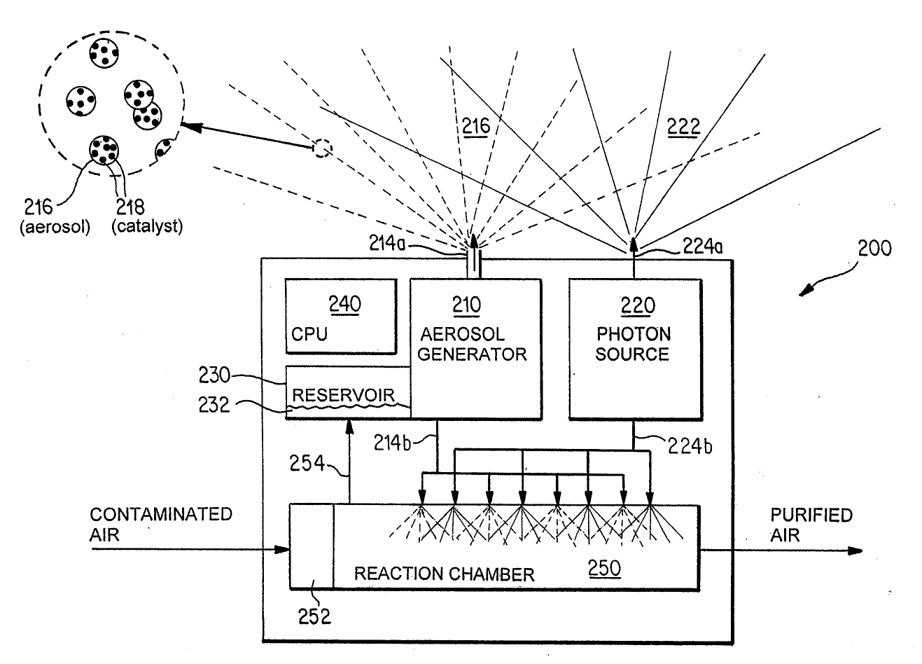

### BACKGROUND OF THE INVENTION

Photocatalysis, which is the acceleration of a photoreaction by the presence of a catalyst, can be used to eliminate organic contaminants such as bacteria, hydrocarbons, and volatile organic compounds (VOCs) from liquid, gaseous, and solid media. Advantageously, photocatalysis is a simple, low cost, and rapid process and is attractive for a variety of purification applications.

Conventional photocatalysis-based apparatus for the elimination of organic contaminants typically use a substrate to support the photocatalytic material. Because the catalytic material is supported on a fixed support and the media to be purified must be brought into contact with the catalyst. A disadvantage of those conventional apparatus is its inability to treat immobile media, such as a surface that has been contaminated by organic or microbial materials. Further, in the example where the media to be purified is a gas or an open volume, another disadvantage is the requirement that the contaminated gaseous media itself must be circulated in order to expose the contaminant to the supported catalyst.

In view of the foregoing, it is one object of the present invention to provide an efficient photocatalytic system and method for decontaminating surfaces as well as small and large gas volumes.

It is an additional object of the present invention to provide a photocatalytic system that is readily employable as a stand-alone air or surface decontamination unit whereby the photocatalyst is brought into contact with the media to be purified.

It is a further object of the invention to provide an inexpensive, non-toxic, easy-to-use system for purifying and/or sterilizing liquid, gaseous and solid media. Such a system is preferably compatible with existing filtration and purification systems.

These and other objects and advantages of the invention are achieved by a disseminating device for producing an artificial fog (i.e., aerosol) that, when exposed to radiation of a suitable wavelength, photocatalytically removes volatile organic compounds and other organic substances from the air and surrounding surfaces. Specifically, the artificial fog according to the invention can oxidize and break down volatile organic compounds, bioaerosols and other organic materials that contaminate the air and surrounding surfaces, resulting in their purification and/or sterilization. In addition to purifying and/or sterilizing contaminated media, the claimed system can advantageously reduce or eliminate odors resulting from volatile organic contaminants and microorganisms that are found in indoor air.

The disseminating device includes a nebulizer or aerosol generator for forming and dispersing an aerosol comprising photocatalytic material, and a photon source adapted to illuminate the dispersed aerosol with electromagnetic radiation having a flux and photon energy effective to initiate photocatalytic reaction.

The artificial fog comprises aerosolized particles that, in turn, comprise particles of a photocatalytic material. Preferably, the photocatalytic particles are nanoscale particles. By providing aerosolized particles that are laden with nanoscale photocatalytic particles, the overall reaction rate can be enhanced by virtue of the high surface area (ratio of surface area to volume) of the particles available for interaction and catalysis.

According to a further embodiment, the invention relates to a method for eliminating organic contaminants from liquid, gaseous and solid media. The method comprises producing an aerosolized medium which carries nanoscale particles of a photocatalytic material, introducing the nanoscale photocatalytic material to the contaminated media, and irradiating the photocatalytic material with photons to initiate the photocatalytic oxidation of contaminants within or on the contaminated media.

Other objects, advantages and novel features of the present invention will become apparent from the following detailed description of the invention when considered in conjunction with the accompanying drawings wherein like elements are indicated by like numerals.

### BRIEF DESCRIPTION OF THE DRAWINGS

The single FIGURE is an illustration of a photocatalytic fog disseminating system according to one embodiment of the invention.

### DETAILED DESCRIPTION OF THE DRAWINGS

The invention relates to a system and method for the photocatalytic oxidation of organic contaminants. Various embodiments according to the present invention are described below with reference the FIGURE, which schematically illustrates a photocatalytic fog disseminating system according to the present invention.

The claimed system 200 comprises an aerosol generator or nebulizer for producing and dispersing an aerosolized photocatalytic material 210 and a photon source 220 having an output that catalytically activates the aerosolized photocatalytic material. Such nebulizers or aerosol generating devices are well known to those skilled in the art, and may be provided, for example, in the form of a conventional atomizer that disperses a liquid into small aerosols, and produces a fog. Any nebulizer that transforms a liquid into particles a few microns in size would be suitable, so that the aerosol mist or fog can easily disperse.

The system 200 forms an aerosol 216 of catalytic particles 218 that are ejected from outlets 214a or 214b of the device. Once formed, the aerosol of catalytic particles can be dispersed into the surroundings using natural or forced convection.

According to a preferred embodiment, the photocatalytic fog disseminating system 200 forms an aerosol 216 from an aqueous suspension 232 of nanoscale photocatalytic particles. The aqueous suspension can be prepared by mixing particles of the photocatalytic material 218 with a liquid and dispersing the particles within the liquid to form a homogeneous suspension. The photocatalytic particles can be dispersed using, for example, a sonicator (that is, an ultrasonicating device). The photocatalytic fog disseminating system 200 preferably includes a reservior 230 of a renewable or replaceable suspension 232 of photocatalytic particles.

The liquid used to form the aqueous suspension may be distilled water that is free of particles greater in size than about 1 micron. An alternative aqueous solution could also be water with an appropriate surfactant or a buffered solution.

If a dry aerosol is desired, the photocatalytic fog disseminating system can be provided with a drying tower; or an aqueous aerosol with an electrostatic neutralization technique could be used to keep the particles from sticking to each other, or to charged surfaces.

The aerosol particles formed using the photocatalytic fog disseminating system preferably range in size of a few microns or less so they stay suspended in the air. Each aerosol particle or agglomeration of particles 216 comprises a plurality of photocatalyst particles 218, as depicted in the FIGURE.

Once dispersed, and while airborne and/or after they have landed on a surface, the photocatalytic particles 218 can be catalytically activated using a photon source 220, which includes output windows 224a and 224b. The inventive system comprises at least one photon source 220 that is adapted to irradiate the artificial fog as it is dispersed through or onto the media to be purified. Examples of suitable photon sources include sunlight, UV lamps (e.g., black lights), light emitting diodes (LEDs), or other similar light sources. The photon source preferably emits electromagnetic radiation 222 having a sufficient flux and quantum energy to catalytically excite the photocatalytic material.

The photocatalytic fog disseminating system can be operated manually or automatically. For instance, a user can simply turn on the photocatalytic fog disseminating system in response to a known need. Alternatively, automatic operation of the photocatalytic fog disseminating system can include automatic triggering of the system using a remote sensor (not shown). Such a sensor can detect air or surface conditions and, via a controller or central processing unit 240 that is incorporated into the system 200, turn the system on and off as required.

According to a preferred method of operation, control of the aerosol dispensing function and control of the photon irradiation function are coupled such that each operates in conjunction with the other. That is, according to one embodiment, the photon source 220 is automatically turned on during the period when the photocatalytic fog is generated, and the photon source is turned off when fog generation is stopped such that electromagnetic radiation 222 is incident on the photocatalyst particles 218 as they come into contact with the media to be purified.

According to another preferred embodiment, the photocatalytic fog disseminating system can also include a reaction chamber 250, and the aerosol 212 can be dispensed into the reaction chamber 250 via outlet 214b such that the aerosol is not dispersed into the open surroundings. In this way, the components of the aerosol (liquid and catalyst particles) can be condensed (via a condenser, not shown), collected and recycled via conduit 254, and the nebulizer (aerosol generator) 210. An air filter may be added on the input side and output side of the chamber to prevent particles from entering the chamber and the photocatalytic fog from leaving the chamber for the enclosed implementation.

Titanium dioxide (titania) is preferred as the sole photocatalytic material, although other such photocatalytic materials may be suitable as well. In particular, intrinsic or doped titanium dioxide anatase phase nanoparticles with a size distribution of less than 10's of nanometers may be used. The nanoscale particle size assists in achieving a uniform dispersion throughout the aqueous solution and in maximizing the efficiency of the photocatalytic reaction. The nano scale particles can also be easily suspended within the aerosols because they are much smaller than the aerosols. When used as catalysts, for example, the catalytic activity of nanoscale particles is expected to be enhanced due to an increased surface area as well as the contribution of surface properties such as surface defects.

The photocatalytic fog disseminating system has a variety of uses, which include, but are not limited to, the purification of air in contaminated buildings, sometimes known as “sick building syndrome,” decontamination of both enclosed and open areas, and the sterilization of medical and agricultural facilities. Examples include hospital patient rooms, operating rooms, chicken coops, food or grain storage facilities, airplane cabins, railroad cars, subways, and hulls of ships. The photocatalytic fog disseminating system can be used in response to hazardous gas releases, or to treat areas after exposure to chemical or biological weapons.

For purifying or sterilizing air, the artificial fog can be dispersed into large or small open volumes, or as described above, the artificial fog can be used in conjunction with a closed reaction chamber such that it is not released into the environment. In the example of a closed reaction chamber, the air to be purified can be forced through the reaction chamber while the fog is irradiated therein. Means for circulating the air to be purified through the reaction chamber include a sub-system of one or more fans.

The foregoing disclosure, including the drawings, has been set forth merely to illustrate the invention and is not intended to be limiting. Since modifications, combinations and sub-combinations of the disclosed embodiments incorporating the spirit and substance of the invention may occur to persons skilled in the art, the invention should be construed to include everything within the scope of the appended claims and equivalents thereof.

What is claimed is:

1. A photocatalytic fog disseminating system for purifying media contaminated with organic impurities, said system comprising:
means for forming and dispersing an aerosol comprising nanoscale photocatalytic particles, and
a photon source adapted to irradiate the dispersed aerosol with electromagnetic radiation.

2. The photocatalytic fog disseminating system according to claim 1, wherein said means for forming and dispersing an aerosol comprises a reservior adapted to hold a renewable or replaceable suspension of photocatalytic particles.

3. The photocatalytic fog disseminating system according to claim 1, wherein said photon source is operatively configured to irradiate the dispersed aerosol with photons having a flux and energy effective to initiate photocatalytic reactions between the photocatalytic particles and the organic impurities.

4. The photocatalytic fog disseminating system according to claim 1, wherein the photon source is selected from the group consisting of sunlight, UV lamps, and light emitting diodes.

5. The photocatalytic fog disseminating system according to claim 1, wherein the aerosol particles disperse the light from the photon source.

6. The photocatalytic fog disseminating system according to claim 1, further comprising:
a reaction chamber into which the aerosol is dispersed; and
a circulation device which circulates air to be purified through said reaction chamber.

7. The photocatalytic fog disseminating system according to claim 1, wherein the system is operatively configured to recycle the aerosol that is dispersed into said reaction chamber.

8. The photocatalytic fog disseminating system according to claim 1, wherein the aerosol and photon source are emitted into the open air outside the system.

9. The photocatalytic fog disseminating system according to claim 1, further comprising a sensor adapted to detect air or surface conditions and, automatically turn the system on and off.

10. A method for purifying media contaminated with at least one of organic and microbe impurities, said method comprising:
forming and dispersing an aerosol comprising nanoscale photocatalytic particles;
contacting the dispersed aerosol with the contaminated media; and
irradiating the photocatalytic particles with photons having a flux and energy effective to initiate photocatalytic reactions between the photocatalytic particles and the at least one of organic and microbe impurities in order to reduce the concentration of said at least one of organic and microbe impurities.

11. The method according to claim 10, further comprising:
forming an aqueous suspension of the nanoscale photocatalytic particles in a liquid, and
forming the aerosol of the photocatalytic particles from the aqueous suspension.

12. The method according to claim 10, wherein the aqueous suspension comprises an additive selected from the group consisting of surfactants and buffering agents.

13. The method according to claim 10, wherein the dispersed aerosol has a particle size on the order of microns or below.

14. The method according to claim 10, further comprising:
directing air to be purified into a reaction chamber;
dispersing the aerosol into the reaction chamber; and
irradiating the photocatalytic particles within the reaction chamber.

15. The method according to claim 14, further comprising:
condensing the aerosol within the reaction chamber and recycling the liquid and the photocatalytic particles.

16. The method according to claim 10, wherein the photocatalytic particles are irradiated while airborne.

17. The method according to claim 10, wherein the photocatalytic particles are irradiated after having landed onto a surface.

18. The method according to claim 10, wherein the dispersing and the irradiating are conducted simultaneously.

19. The method according to claim 10, wherein the photocatalytic particles consist essentially of titanium oxide on the order of microns or below.

20. The method according to claim 19, wherein the titanium dioxide consists essentially of anatase.

21. The method according to claim 10, wherein the photocatalytic particles comprise an intrinsic semiconductor material.

22. The method according to claim 10, wherein the photocatalytic particles comprise a doped semiconductor material.
